(19) United States
(12) Patent Application Publication
(10) Pub. No.: US 2026/0185024 A1
(43) Pub. Date: Jul. 2, 2026

(54) TISSUE ENGINEERING SCAFFOLDS

(71) Applicant: VESTLANDETS INNOVASJONSSELSKAP AS, Bergen (NO)

(72) Inventors: Ahmad Rashad Saad Mohamed Elsebahy, Ankara (TR); Kamal Babikeir Elnour Mustafa, Bergen (NO); Adel Badria, Ankara (TR)

(21) Appl. No.: 19/121,689

(22) PCT Filed: Oct. 18, 2023
(86) PCT No.: PCT/EP2023/079067

§ 371 Date: Apr. 16, 2025

(30) Foreign Application Priority Data
Oct. 20, 2022 (GB) 2215542.8

(51) Int. Cl. C12M 3/00; A61F 2/30; C12M 1/12

(52) U.S. Cl. CPC C12M 21/08; A61F 2/30942; C12M 23/46; C12M 25/14

(57) ABSTRACT

A tissue engineering scaffold assembly comprises a plurality of corresponding tissue engineering scaffolds. Each scaffold comprises an inner portion comprising a first set of one or more walls defining a channel extending along an axial direction from a first end to a second end of the scaffold and an outer portion comprising a second set of one or more walls substantially surrounding the first set of walls with a spacing between them defining a cavity. Each scaffold also includes a first axial interlocking part arranged at the first end of the scaffold, extending in the axial direction beyond the first and/or second set of one or more walls and a second axial interlocking part arranged at the second end of the scaffold. The second axial interlocking part of one of the plurality of corresponding tissue engineering scaffolds is engaged with the first axial interlocking part of another of the plurality of corresponding tissue engineering scaffolds to create an interlock in the axial direction.

### BACKGROUND OF THE INVENTION

The present invention relates to devices and methods for tissue engineering using at least one tissue engineering scaffold, and in particular to the physical structure of the tissue engineering scaffolds.

Tissue engineering generally relates to the growth of new tissues, or organs, from living cells using a scaffold as a support during growth, either in vitro or in vivo. Tissue engineering techniques may be used to produce an organ or tissue graft for implantation back into a donor host. Tissue engineering frequently involves stem cells; implanting stem cells in an appropriate location can generate bone, tendon and cartilage. Applications include dermal wound healing and repair of cartilage, ligament or bone.

Bone regeneration or repair typically involves the implantation of a bone graft. However, the failure rate of bone grafts can be high. In bone tissue engineering, the osteogenic and angiogenic potential of stem cells in 3D structural systems has been demonstrated in vitro and in vivo. A classic tissue engineering treatment uses bone cells carried by a synthesized scaffold to accelerate healing procedures.

It is desirable that bone scaffolds be customizable to some degree, since scaffolds having different dimensions or different properties can be more suitable for particular purposes, depending on the site of implantation, and the extent of bone damage, amongst other factors. It is known, for example, to scan a patient's bone and construct an individualised scaffold based on their particular bone dimensions. However, individual customisation is expensive and time-consuming.

There are also various challenges to creating such scaffolds. For example, such scaffolds can be produced using known 3D printing techniques, however thermal degradation of thermoplastic polymeric materials occurs in the print material, leading to inconsistency in the parts of the scaffold which are printed later in the extrusion-based printing process. This is particularly problematic where long-bone scaffolds are printed, since the scaffolds need to have a large length in order to be suitable for growing a replacement long bone.

There remains a need for tissue engineering scaffolds that address some of these shortcomings.

### SUMMARY OF THE INVENTION

From a first aspect, the invention provides a tissue engineering scaffold comprising:
an inner portion comprising a first set of one or more walls defining a channel extending along an axial direction from a first end of the scaffold to a second end of the scaffold;
an outer portion comprising a second set of one or more walls and arranged such that the second set of one or more walls substantially surrounds the first set of one or more walls with a spacing between the first and second sets of one or more walls defining a cavity between the inner portion and the outer portion;
a first axial interlocking part arranged at the first end of the scaffold, extending in the axial direction beyond the first set of one or more walls and/or the second set of one or more walls; and
a second axial interlocking part arranged at the second end of the scaffold to engage with the first axial interlocking part of another corresponding tissue engineering scaffold to create an interlock in the axial direction.

The skilled person will understand that multiple corresponding scaffolds might therefore be engaged with each other, so as to interlock together, giving a tissue engineering scaffold assembly comprised of multiple scaffolds.

Thus, from a second aspect, the invention provides a tissue engineering scaffold assembly comprising:
a plurality of corresponding tissue engineering scaffolds, wherein each scaffold comprises:
an inner portion comprising a first set of one or more walls defining a channel extending along an axial direction from a first end of the scaffold to a second end of the scaffold;
an outer portion comprising a second set of one or more walls and arranged such that the second set of one or more walls substantially surrounds the first set of one or more walls with a spacing between the first and second sets of one or more walls defining a cavity between the inner portion and the outer portion;
a first axial interlocking part arranged at the first end of the scaffold, extending in the axial direction beyond the first set of one or more walls and/or the second set of one or more walls; and
a second axial interlocking part arranged at the second end of the scaffold;
wherein the second axial interlocking part of one of the plurality of corresponding tissue engineering scaffolds is engaged with the first axial interlocking part of another of the plurality of corresponding tissue engineering scaffolds to create an interlock in the axial direction.

Thus it will be seen that, in accordance with the invention, by providing a scaffold with both a first axial interlocking part and a second axial interlocking part, it is possible for multiple such parts to be interlocked together (i.e. stacked) in the axial direction, to produce a tissue engineering scaffold assembly having a greater length in the axial direction. As a result, the total length of the scaffold in the axial direction is customisable by choosing how many scaffolds to interlock. This means that many different lengths of scaffold assembly, useful for different applications and implantation sites, can be produced from the same (or at least corresponding) scaffolds. This is helpful since information about the particular implantation site or bone defect does not need to be known at the time the scaffold is produced. It will be understood by “corresponding” that each scaffold does not need to be entirely identical with the other scaffolds in a given assembly, rather they need only correspond to the extent required in order for them to be able to interlock. In other words, the first axial interlocking part of one scaffold corresponds with the second axial interlocking part of the other scaffold, and vice versa. Scaffolds made of different materials or with other differing properties (e.g. different materials, or with some parts having different shapes or sizes) could still be “corresponding” provided that their respective axial interlocking parts are arranged to interlock. In some embodiments, some or all of the scaffolds in an assembly may be identical.

Furthermore, by being able to stack the scaffolds, to produce a longer scaffold assembly, each individual scaffold can be made shorter in the axial direction. This makes the individual scaffolds easier to manufacture, firstly since many identical or at least corresponding scaffolds can be produced, rather than having to produce scaffolds of different shapes and sizes for different functions. Additionally, a smaller axial size is particularly advantageous where the scaffold is to be 3D-printed, since greater control of the printing process is possible when printing smaller parts and thermal degradation of the printing material is reduced since the printing time is shorter.

It will be understood that the “interlocking” of the scaffolds by the axial interlocking parts requires only that the scaffolds are interlocked in the axial direction, i.e. so as to prevent, restrict or resist relative movement between the scaffolds in the axial direction. Whilst the interlock could optionally restrict relative movement between the scaffolds in other directions, e.g. in all directions, the only requirement is that the interlock prevents movement in the axial direction.

By the statement that the second set of one or more walls substantially surrounds the first set of one or more walls, the skilled person will understand that the second set of walls extends around, or covers, approximately all of the first set of walls, i.e. they extend around approximately the same angular range. The second set of one or more walls is not required to fully enclose the first set of one or more walls. It will furthermore be understood that since the first set of one or more walls is not necessarily closed, i.e. extending around an angular range of 360 degrees or less, the channel defined by the first set of one or more walls is not necessarily closed. It may be an open shape, e.g. with a semi-circular cross-section.

Any of the angular ranges referred to herein may be defined as an angular range around the axial direction. In other words, the angular range may be defined along a “circumferential direction” which lies in a plane perpendicular to the axial direction.

In some embodiments, the first and second sets of one or more walls substantially extend across an angular range of 360 degrees i.e. full circle, thus the channel is fully enclosed in directions other than the axial direction. The first set of one or more walls and/or the second set of one or more walls may be continuous through 360 degrees, or alternatively could also be formed by multiple separate pieces which together span 360 degrees.

Alternatively, in other embodiments the first set of one or more walls extends across an angular range of less than 360 degrees, such that there is an opening in the first set of one or more walls. Alternatively, or in addition, the second set of one or more walls may extend across an angular range of less than 360 degrees, such that there is an opening in the second set of one or more walls. It will be understood that this opening extends across the entire axial extent of the walls, i.e. there is an angular range around the axial direction in which no wall is present.

Providing an opening in both sets of walls allows the central channel to be open to an external environment in which the scaffold is placed. During implantation, existing structure at the implantation site, such as nerves or blood vessels, can be moved through the opening into the open channel, thus moving the existing structure to inside the scaffold, without having to interfere with the existing structure, e.g. no need to sever the nerve so that it can be passed through the channel.

Providing a scaffold in which both the first and second set of one or more walls contain an opening, such that existing structure may be placed more easily inside the channel via the opening, is considered to be novel and inventive in its own right. Thus, from a third aspect, there is provided a tissue engineering scaffold comprising:
an inner portion comprising a first set of one or more walls defining a channel extending along an axial direction from a first end of the scaffold to a second end of the scaffold;
an outer portion comprising a second set of one or more walls and arranged such that the second set of one or more walls substantially surrounds the first set of one or more walls with a spacing between the first and second sets of one or more walls defining a cavity between the inner portion and the outer portion;
wherein the first and second sets of one or more walls extend across an angular range of less than 360 degrees such that the channel is open in a direction other than the axial direction.

In some embodiments, such a tissue engineering scaffold may further comprise a first axial interlocking part arranged at the first end of the scaffold, extending in the axial direction beyond the first set of one or more walls and/or the second set of one or more walls; and a second axial interlocking part arranged at the second end of the scaffold to engage with the first axial interlocking part of another corresponding tissue engineering scaffold to create an interlock in the axial direction.

In some embodiments the opening in the first set of walls and the opening in the second set of walls are aligned. This makes insertion of a structure into the channel, e.g. during implantation, easier and more convenient.

In some embodiments the first and second sets of one or more walls extend across an angular range of approximately 180 degrees, or more than 180 degrees. Optionally, the first and second sets of one or more walls extend across an angular range of more than 270 degrees. This is particularly advantageous because, by having the walls extend around a greater angular range their strength is increased, and less, or no, structural support might be needed across the opening of the scaffold.

In some embodiments in which the second sets of one or more walls (and optionally also the first set of one or more walls) extend across an angular range of less than 360 degrees, such that there is an opening in the second set of walls (and optionally the first set of walls), the tissue engineering scaffold may further comprise a first radial interlocking part and a second radial interlocking part arranged to engage with the first radial interlocking part of another corresponding tissue engineering scaffold to create an interlock in the radial direction, perpendicular to the axial direction.

This allows two corresponding scaffolds to be attached together in the radial direction, e.g. two semi-circular halves to be connected to form a complete circle. Thus the two scaffolds could be arranged on either side of an existing structure, such as a nerve, so that the nerve sits within the channel, and then the two scaffolds could be radially interlocked together. In some embodiments, the second set of walls may have an angular range of about 180 degrees. However, as defined above, two “corresponding” scaffolds need not be identical, and it will therefore be understood in this context that the scaffolds need only be shaped to interlock in the radial direction. For example, they could have different shapes. One scaffold could extend across 270 degrees and the other could extend across 90 degrees, such that when interlocked they form a scaffold structure having a total angular range of 360 degrees, i.e. around the axial direction.

Providing a scaffold having first and second radial interlocking parts which allow the scaffold to interlock with another corresponding scaffold to create a radial interlock, is considered to be novel and inventive in its own right. Thus, from a fourth aspect, there is provided a tissue engineering scaffold comprising:
an inner portion comprising a first set of one or more walls defining a channel extending along an axial direction from a first end of the scaffold to a second end of the scaffold;
an outer portion comprising a second set of one or more walls and arranged such that the second set of one or more walls substantially surrounds the first set of one or more walls with a spacing between the first and second sets of one or more walls defining a cavity between the inner portion and the outer portion;
wherein the second set of one or more walls extends across an angular range of less than 360 degrees; and
wherein the tissue engineering scaffold comprises a first radial interlocking part arranged to engage with the second radial interlocking part of another corresponding tissue engineering scaffold to create an interlock in the radial direction, perpendicular to the axial direction.

The tissue engineering scaffold may further comprise a second radial interlocking part, which may be arranged to engage with a first radial interlocking part of another corresponding tissue engineering scaffold.

A scaffold structure may be formed by radially interlocking two such scaffolds, thus according to a fifth aspect there is provided a scaffold structure comprising:
a first scaffold and a second scaffold, wherein each scaffold comprises:
an inner portion comprising a first set of one or more walls defining a channel extending along an axial direction from a first end of the scaffold to a second end of the scaffold; and
an outer portion comprising a second set of one or more walls and arranged such that the second set of one or more walls substantially surrounds the first set of one or more walls with a spacing between the first and second sets of one or more walls defining a cavity between the inner portion and the outer portion;
wherein the second set of one or more walls extends across an angular range of less than 360 degrees; and
wherein the first scaffold comprises a first radial interlocking part and the second scaffold comprises a second radial interlocking part;
wherein the first radial interlocking part of the first scaffold is engaged with the second radial interlocking part of the second scaffold to create an interlock in the radial direction, perpendicular to the axial direction.

In a similar way to the axial interlocking described above, by forming a scaffold structure from at least two separate scaffolds there can be more flexibility in the form of the overall structure, and the individual scaffolds may be easier to manufacture.

As laid out above, in some embodiments the first set of one or more walls also extends across an angular range of less than 360 degrees.

In some embodiments, the radial interlocking may produce a scaffold structure which is open, e.g. in which the central channel is open, and/or in which the walls of the inner portion and/or the walls of the outer portion are not continuous. The total angular range of the second sets of one or more walls in the scaffold structure may be less than 360 degrees.

In some other embodiments, the radial interlocking may produce a scaffold structure which is closed, e.g. in which the central channel is closed, and/or in which the walls of the inner portion and/or the walls of the outer portion are continuous. The total angular range of the second sets of one or more walls in the scaffold structure may be 360 degrees.

In some embodiments the first and second scaffolds interlock with each other at both ends of their angular range. For example, the first scaffold may further comprise a third radial interlocking part and the second scaffold may further comprise a fourth radial interlocking part, wherein the third radial interlocking part of the first scaffold is engaged with the fourth radial interlocking part of the second scaffold to create another interlock in the radial direction.

It will be understood that a series of such scaffold structures, optionally having axial interlocking parts as described above, could also be stacked in the axial direction to produce a scaffold assembly.

In some embodiments the first (and optionally the fourth) radial interlocking part is provided by one or more teeth, and the second (and optionally the third) radial interlocking part is provided by one or more recesses, or vice versa. The first radial interlocking part and/or the second radial interlocking part may extend perpendicular to the axial direction, i.e. along a direction which is tangential to the circumferential direction.

The first radial interlocking part and/or the second radial interlocking part may be arranged within the length of the channel, i.e. between the first end and the second end of the first/second scaffold. The first radial interlocking part and/or the second radial interlocking part may extend along substantially the length of the channel. Where provided, the third radial interlocking part and/or the fourth radial interlocking part may be arranged within the length of the channel, i.e. between the first end and the second end of the first/second scaffold. The third radial interlocking part and/or the fourth radial interlocking part may extend along substantially the length of the channel.

In some embodiments, the first and/or second scaffold further comprises a connecting portion, extending along the axial direction, and connecting the outer portion and the inner portion, such that an edge of the opening of the inner portion is connected to an edge of the opening of the outer portion by the connecting portion. This provides structural support to the first and second sets of walls, in the region near the opening, and also provides some degree of closure of the cavity.

In some embodiments, the first and/or second scaffold further comprises a second connecting portion, extending along the axial direction, and connecting the outer portion and the inner portion, such that a second edge of the opening of the inner portion is connected to a second edge of the opening of the outer portion by the second connecting portion.

The first connecting portion may provide the first radial interlocking part. The second connecting portion may provide the second radial interlocking part. This conveniently utilises existing parts of the scaffold to provide radial interlocking, thus avoiding occupying space in the channel and the cavity and additionally minimising the additional material required for producing the scaffold.

The first connecting portion and/or the second connecting portion may comprise windows, allowing flow into and/or out of the cavity.

As mentioned above, a scaffold structure formed from radially interlocked first and second scaffolds may then act as a tissue engineering scaffold having features according to the first aspect of the invention. Some further features of a tissue engineering scaffold or scaffold assembly according to any of the aspects will now be described.

In some embodiments, one of the first axial interlocking part and the second axial interlocking part is a male part, and the other of the first axial interlocking part and the second axial interlocking part is a female part, sized and shaped to receive the male part. This provides a reliable axial interlocking mechanism. In some embodiments, the male part is cylindrical, e.g. a full cylinder, or the partial axial extent of a cylinder, and the female part is a corresponding groove or recess of slightly larger circumference than the cylinder.

In some embodiments, the first set of one or more walls comprises the first axial interlocking part and/or the second axial interlocking part. In some embodiments the second set of one or more walls comprises the first axial interlocking part and/or the second axial interlocking part. Optionally one of the sets of walls may comprise both the first and the second axial interlocking parts. This conveniently utilises existing parts of the scaffold to provide axial interlocking, thus avoiding occupying space in the channel and the cavity. This is also relatively easy to manufacture since a part can simply be added to, or removed from, the existing set of walls during manufacture. Thus the first axial interlocking part and the second axial interlocking part may have the same shape (i.e. cross-sectional shape) as the inner portion or the outer portion (and may therefore have any of the shapes described below).

In some embodiments the second axial interlocking part is sized to entirely accommodate the portion of the first axial interlocking part which extends in the axial direction beyond the first set of one or more walls and/or the second set of one or more walls, such that when interlocked in the axial direction the tissue engineering scaffold will be flush with another corresponding tissue engineering scaffold to which it is interlocked. This results in a scaffold assembly in which the inner and outer walls are substantially continuous in the axial direction, e.g. allowing a bone produced using the scaffold to have improved mechanical properties.

In some embodiments, the first set of one or more walls and/or the second set of one or more walls are solid, non-porous walls, comprising one or more apertures. This allows material to flow through the sets of walls in the radial direction to get into/out of the cavity and the channel.

In some embodiments the apertures comprise a chamfered or bevelled edge, i.e. a sloped edge. This provides a shape which is more easily 3D printed, and has improved mechanical properties because it reduces stress concentration and prevents collapsing of the edges under compression. Moreover, the chamfered or bevelled edge of the apertures decreases the sagging of the hot printed filaments traveling in the air between the ends of the apertures, during printing. The apertures may be pentagonal, e.g. be a pentagon, i.e. defining internal angles that comprise two right angles, one acute angle and two obtuse angles.

In some embodiments the first set of one or more walls and/or the second set of one or more walls comprise more than one wall. Thus, in some embodiments the inner portion comprises a first wall and a second wall (e.g. with a space between them), wherein the first wall and the second wall are concentric. In some embodiments, in addition or alternatively, the outer portion comprises a first wall and a second wall (e.g. with a space between them), wherein the first wall and the second wall are concentric. This provides a structure for the inner portion/outer portion in which there are two concentric walls, or layers, with a gap between them. This helps to increase the mechanical strength of the inner/outer portion.

Providing a bone scaffold having an inner portion and/or an outer portion provided by two concentric walls with a gap between them is considered to be novel and inventive in its own right. Thus, from a sixth aspect, there is provided a tissue engineering scaffold comprising:
an inner portion comprising a first set of one or more walls defining a channel extending along an axial direction from a first end of the scaffold to a second end of the scaffold;
an outer portion comprising a second set of one or more walls and arranged such that the second set of one or more walls substantially surrounds the first set of one or more walls with a spacing between the first and second sets of one or more walls defining a cavity between the inner portion and the outer portion;
wherein the inner portion and/or the outer portion comprise more than one concentric wall.

The first wall and the second wall may each comprise apertures. The apertures in the first and second wall may be aligned. This allows radial flow between the cavity and an external environment (where the apertures are in the outer portion) and between the cavity and the channel (where the apertures are in the inner portion).

In some embodiments the tissue engineering scaffold further comprises struts, extending between the first wall and the second wall, wherein the struts are spaced apart in both the axial direction and in the circumferential direction, thereby allowing flow in both the axial and circumferential directions.

The inner and/or outer portion (and optionally the entire scaffold) may be formed from any biomaterial (e.g., metals such as titanium and ceramics such as hydroxyapatite or composite of them). In some embodiments, the inner and/or the outer portion are formed from polymeric material, e.g. polylactide or polycaprolactone (PCL). A biocompatible and/or bioabsorbable polymeric material may be chosen.

Since the channel extends along the axial direction, it will be understood that at least the inner portion is arranged circumferentially around the axial direction. In various embodiments, the outer portion that surrounds the inner portion is also arranged circumferentially around the axial direction. The second set of walls may be angled relative to the first set of walls, but preferably the second set of walls is parallel to the first set of walls.

In some embodiments the inner portion and the outer portion are concentric (i.e. all of the walls of the first set of one or more walls are concentric with all of the walls of the second set of one or more walls). The outer portion may be arranged symmetrically around the inner portion, e.g. in a rotationally symmetric arrangement.

In some embodiments the inner and outer portion have the same shape, for example they might both be circular. However, even if the inner and outer portion have the same (e.g. circular) shape they may not be arranged concentrically around the axial direction.

In other embodiments, the inner and outer portion have different shapes, i.e. all of the walls of the first set of one or more walls have a different shape to all of the walls of the second set of one or more walls. When the inner and outer portion have different shapes, it may still be possible for both the inner and outer portion to be co-centred.

In some embodiments, the outer portion is circular. In some embodiments the inner portion and/or the outer portion is polygonal, i.e. a shape with corners. Such a shape may be easier to produce, e.g. through 3D-printing, and may also provide improved mechanical properties. For example, where the inner or outer portion provides the first and second axial interlocking parts they may have the same shape as the inner or outer portion and thus a polygonal inner or outer portion will prevent relative rotation of two adjacent scaffolds when they are axially interlocked. The inner and/or outer portion may be a square, a hexagon, or a triangle.

In some embodiments the inner portion further comprises axial conduits. Where the inner portion is polygonal, the axial conduits might be arranged at each corner of the polygonal shape, e.g. between the first and second wall of the inner portion, or inside the second (i.e. inner) wall of the inner portion, thus within the channel. The axial conduits can allow for transport of different materials to the materials travelling through the channel or the cavity.

In some embodiments the length of the scaffold along the axial direction, between the first end and the second end, is approximately equal to, or less than, the width or diameter of the scaffold, perpendicular to the axial direction, i.e. the scaffold is short compared to its width. A short scaffold is easier to manufacture since there is less thermal degradation if the scaffold is 3D-printing, and smaller parts are easier to manufacture.

In some embodiments, the tissue engineering scaffold further comprises a base portion extending between the inner portion and the outer portion, perpendicular to the axial direction. The base portion may extend continuously between the inner portion and the outer portion. It may be a flat plate.

The base portion may be arranged at one end of inner and outer walls, i.e. adjacent to one end of the channel. The other end of the scaffold (i.e. the one at which the base portion is not located) may be open, i.e. with nothing extending between the inner portion and the outer portion at that end.

In some embodiments the base portion comprises or consists of a porous material, for example a mesh, e.g. a micro-filament mesh.

In some embodiments the base portion comprises a window, aligned with the channel. This helps to ensure that the base portion does not restrict or affect the axial flow of material through the channel. Thus the base portion extends only between the inner portion and the outer portion, i.e. the region where support is required for the filler material as discussed below.

According to a seventh aspect, there is provided a tissue engineering device comprising a tissue engineering scaffold assembly or a tissue engineering scaffold as described above, and further comprising a filler material, positioned within the cavity of the or each tissue engineering scaffold. The filler material may be a porous material. In some embodiments, the filler material is a hydrogel material, e.g. cellulose nanofibril (CNF) hydrogel, nanocellulose hydrogel, gelatin-based hydrogel, collagen-based hydrogel, hyaluronic-based hydrogel or any other natural or synthetic shear thinning hydrogels. In other embodiments, the filler material is a ceramic material such as hydroxyapatite, tricalcium phosphate or a combination of both. In other embodiments, the filler material is a composite of ceramic and hydrogel material. In other embodiments, the filler material is a bioink. It will be understood that a bioink is a solution of cells that is printable (e.g., by an automated method). The bioink may comprise biomaterials, such as one or more hydrogels. Other materials may be added to the one or more hydrogels such as mineral phases (e.g., classic ceramics) or other polymers not in a hydrogel form (e.g., nanoparticles, microspheres, fibres). The one or more hydrogels may include or contain tissue forming cells.

In some embodiments the filler material is seeded with tissue precursor cells, e.g. bone cells or stem cells.

It will furthermore be understood that a tissue engineering scaffold assembly may be assembled from a plurality of scaffolds, where various of the scaffolds has different features in accordance with the embodiments laid out above. For example, an assembly may comprise scaffolds containing different filler materials, or constructed of different filler materials. This allows different parts of the assembly to be optimised for their different functions. For example, a tissue engineering scaffold assembly may comprise a plurality of scaffolds wherein the scaffolds positioned at either end of the scaffold assembly have different properties to the one or more central scaffolds.

The described invention further extends to a method of making such scaffolds. Thus, according to an eighth embodiment, there is provided a method of making a tissue engineering scaffold, comprising:
producing a tissue engineering scaffold as described above using a 3D printing method. The 3D printing may be extrusion-based printing or alternatively another printing technique such as fused deposition modelling, stereolithography, selective laser sintering, or direct metal laser sintering. Thus the scaffolds described above may be printed by an extrusion-based printer, fused deposition modelling, stereolithography, selective laser sintering, or direct metal laser sintering.

While the description above relates to tissue engineering scaffolds in a general sense, it will be appreciated that the scaffolds, assemblies and structures described herein are particularly well-suited to supporting bone growth. Hence, in any aspect or embodiment described herein the scaffold may be designated as a bone growth scaffold, most preferably a scaffold for long bone growth. Long bones are those that are longer than they are wide. In humans, the long bone category includes the femora, tibiae, and fibulae of the legs; the humeri, radii, and ulnae of the arms; metacarpals and metatarsals of the hands and feet, the phalanges of the fingers and toes, and the clavicles or collar bones.

Features of any aspect or embodiment described herein may, wherever appropriate, be applied to any other aspect or embodiment described herein. In particular, although features may be described following a statement concerning one particular “aspect” of the invention, it will be understood that these features may be present likewise in other “aspects”. Where reference is made to different embodiments or sets of embodiments, it should be understood that these are not necessarily distinct but may overlap.

### BRIEF DESCRIPTION OF THE DRAWINGS

Certain preferred embodiments of the invention will now be described, by way of example only, with reference to the accompanying drawings, in which:

FIG. 1 is a perspective view of a scaffold according to a first embodiment of a first aspect of the present invention;

FIG. 2 is a perspective view of the scaffold of FIG. 1, showing the scaffold filled with a filler material;

FIG. 3 is a perspective view of the scaffold of FIG. 1, omitting the first axial interlocking part to show the rest of the scaffold more clearly;

FIG. 4 is a side view of the scaffold of FIG. 1;

FIG. 5 is a cross-sectional side view, taken along the line A-A shown in FIG. 4;

FIG. 6 is a view from above of the scaffold of FIG. 1;

FIG. 7 is a perspective view showing a scaffold assembly, according to a first embodiment of a second aspect of the present invention, comprised of a plurality of scaffolds, as shown in FIG. 1;

FIG. 8 is a perspective view showing a scaffold assembly, according to a second embodiment of a second aspect of the present invention, comprised of a plurality of scaffolds according to a second embodiment of a first aspect of the present invention;

FIG. 9 is a view from above of a scaffold having a square channel according to a third embodiment of a first aspect of the present invention;

FIG. 10 is a view from above of a scaffold having a hexagonal channel according to a fourth embodiment of a first aspect of the present invention;

FIG. 11 is a view from above of a scaffold having a triangular channel according to a fifth embodiment of a first aspect of the present invention;

FIG. 12 is a view from above of a scaffold having a triangular channel according to a sixth embodiment of a first aspect of the present invention;

FIG. 13 is a perspective view of a scaffold structure, comprising two scaffolds according to a seventh embodiment of a first aspect of the present invention, according to a first embodiment of a third aspect of the present invention, and according to a first embodiment of a fourth aspect of the present invention;

FIG. 14 is a perspective view of one of the scaffolds of the scaffold structure of FIG. 13;

FIG. 15 is a view from above of the scaffold structure of FIG. 13;

FIG. 16 is a side view of the scaffold structure of FIG. 13;

FIG. 17 is a perspective view showing a scaffold assembly, according to a third embodiment of a second aspect of the present invention, comprised of a plurality of the scaffold structures shown in FIG. 13;

FIG. 18 is a perspective view showing a scaffold assembly, according to a fourth embodiment of a second aspect of the present invention, comprised of a plurality of scaffolds as shown in FIG. 19;

FIG. 19 is a perspective view of a scaffold according to an eighth embodiment of a first aspect of the present invention, and according to a second embodiment of a second aspect of the present invention;

FIG. 20 is a perspective view of the scaffold of FIG. 19, showing the scaffold filled with a filler material;

FIG. 21 is a view from above of the scaffold of FIG. 19; and

FIG. 22 is a side view of the scaffold of FIG. 19.

### DETAILED DESCRIPTION

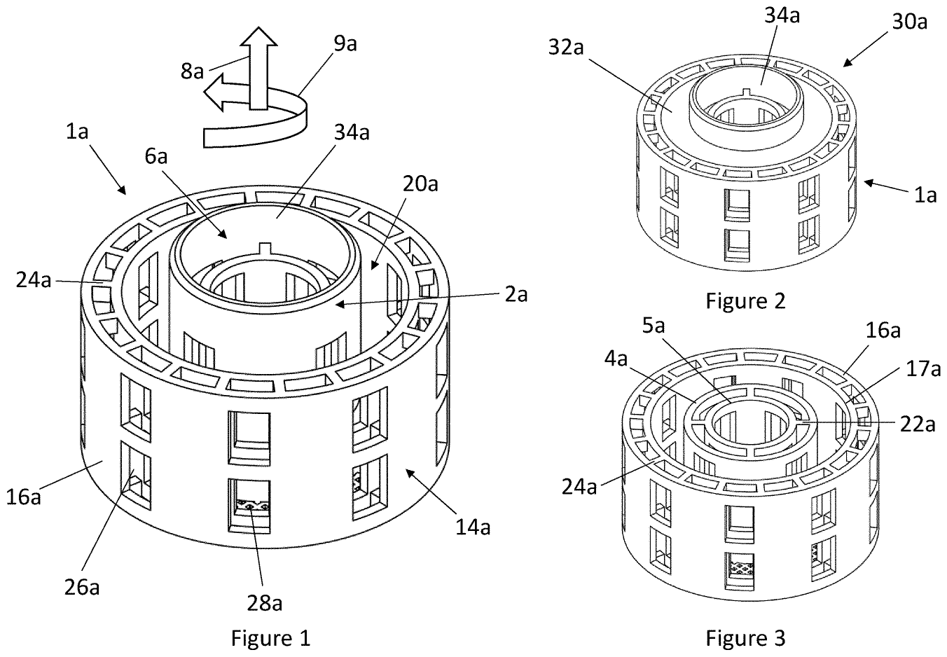

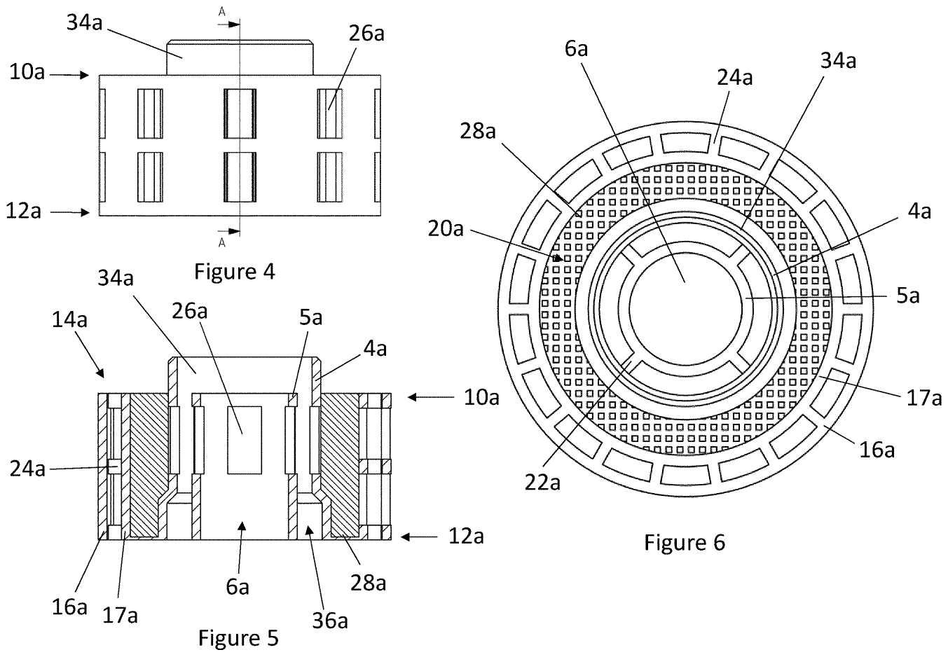

FIGS. 1 to 6 shows a perspective view of a tissue engineering scaffold 1a according to a first embodiment of a first aspect of the present invention. The tissue engineering scaffold 1a comprises an inner portion 2a comprising a first wall 4a and a second wall 5a (seen in FIG. 6) which are spaced apart, i.e. have a gap between them. The inner portion 2a defines a channel 6a, which extends along an axial direction 8a from a first end 10a of the scaffold 1a to a second end 12a of the scaffold 1a.

The scaffold 1a further includes an outer portion 14a, which comprises a first wall 16a and a second wall 17a which are spaced apart, i.e. have a gap between them. The second set of walls 16a, 17a substantially surrounds the first set of walls 4a, 5a, as seen in the view from above of FIG. 6. In this embodiment both sets of walls are circular. The spacing between the first and second sets of walls defines a cavity 20a.

The structure of the inner and outer portions 2a, 14a can be seen more clearly in FIG. 3 in which the first axial interlocking part 34a, described in greater detail below, has been omitted.

There are struts 22a, 24a, extending respectively between the first wall 4a, 16a, and the second wall 5a, 17a, of each of the inner and outer portions 2a, 14a. As seen in the Figures, the struts 22a, 24a are spaced apart in both the axial direction and in a circumferential direction 9a, which surrounds the axial direction 8a and lies in a plane perpendicular to the axial direction, thereby allowing flow of material in both the axial and circumferential directions.

All four walls of the walls 4a, 5a, 16a, 17a are made of a solid, non-porous material. Each of the walls contains apertures 26a, which are open in the radial direction and which allow radial flow into and out of the cavity 20a and the channel 6a.

The scaffold 1a further comprises a base portion 28a, which is positioned at the second end 12a of the scaffold. The base portion 28a is a porous mesh material which extends between the inner portion 2a and the outer portion 14a. There is a window in the base portion 28a such that it does not extend into the channel 6a, as seen in FIG. 6.

The base portion 28a is arranged so that it can support a filler material 32a (seen in FIG. 2) which can be placed inside the cavity 20a. The filler material 32a can be seeded with cells and other materials suitable for growing bone in the cavity. A device 30a comprised of the scaffold 1a, and a filler material 32a placed within the cavity can be seen in FIG. 2.

The scaffold 1a further includes a first axial interlocking part 34a which is arranged at the first end 10a of the scaffold 1a, and extends in the axial direction 8a beyond the walls 16a, 17a of the outer portion 14a. The first axial interlocking part 34a extends as part of the inner portion 2a, in particular as an extension of the first wall 4a, as seen in the cross-sectional view of FIG. 5. The first axial interlocking part 34a is a male part.

The scaffold also includes a second axial interlocking part 36a arranged at the second end 12a of the scaffold 1a, as seen in the cross-sectional view of FIG. 5. In particular, the second axial interlocking part 36a is a circular groove or recess, formed between the first wall 4a and the second wall 5a of the inner portion 2a. The second axial interlocking part 36a is a female part.

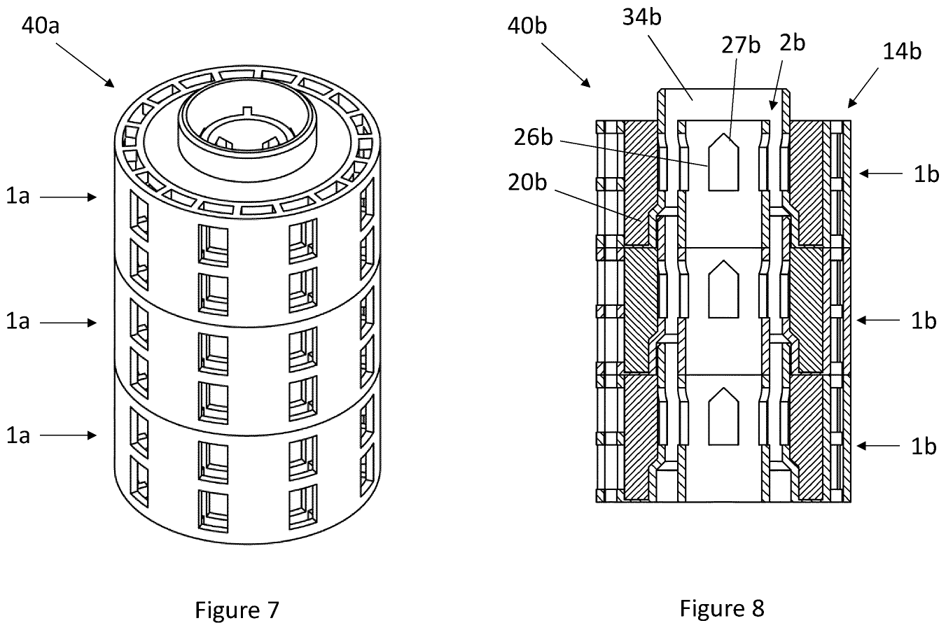

The first axial interlocking part 34a is a cylinder, which is sized to be received into the groove of the second axial interlocking part 36a of another corresponding scaffold. FIG. 7 shows a series of such scaffolds 1a which have been axially interlocked using their respective first and second axially interlocking parts 34a, 36a, to form a tissue engineering scaffold assembly 40a. The arrangement of the first and second axially interlocking parts 34a, 36a allows the scaffolds 1a to be interlocked so as to sit flush against one another in the axial direction, as seen in FIG. 7, giving a continuous structure, i.e. continuous inner and outer portions, channel and cavity.

FIG. 8 shows a second embodiment of a tissue engineering scaffold assembly 40b. Like reference numerals are used for corresponding components of this tissue engineering scaffold assembly 40b as the tissue engineering scaffold assembly 40a of FIG. 7, and the scaffold 1a of FIGS. 1-6, using the same reference numeral but followed by “b”, rather than “a”.

This assembly is stacked in the same manner as the assembly of FIG. 7, with the only difference being that the apertures 26b have a chamfered upper edge 27b. The other components of the scaffold 1b are the same as those of the scaffold 1a described above, and are not described again here.

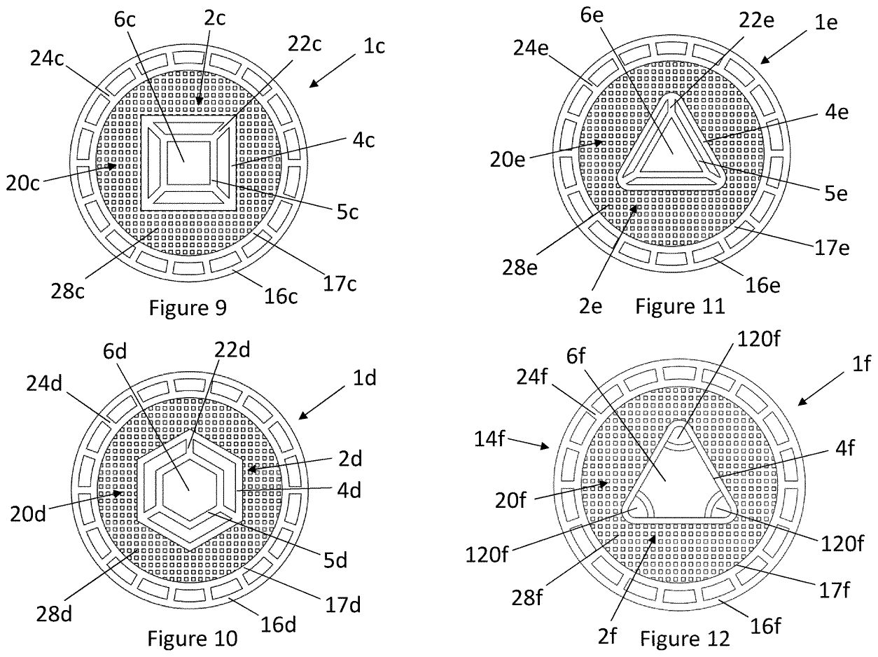

FIG. 9 is a view from above of a third embodiment of a scaffold according to the present invention. The scaffold 1c has an inner portion 2c which is square, and therefore defines a square channel 6c. In all other respects, the scaffold 1c is like the other scaffold 1a which was described above, as is clear from the use of the same reference numerals, but followed by “c”, rather than “a”.

Similarly, FIG. 10 shows a view from above of a fourth embodiment of a scaffold 1d according to the present invention. The scaffold 1d has an inner portion 2d which is hexagonal, and therefore defines a hexagonal channel 6d. In all other respects, the scaffold 1d is like the other scaffold 1a which was described above, as is clear from the use of the same reference numerals, but followed by “d”, rather than “a”.

Again similarly, FIG. 11 shows a view from above of a fifth embodiment of a scaffold 1e according to the present invention. The scaffold 1e has an inner portion 2e which is triangular, and therefore defines a triangular channel 6e. In all other respects, the scaffold 1e is like the other scaffold 1a which was described above, as is clear from the use of the same reference numerals, but followed by “e”, rather than “a”.

FIG. 12 shows a view from above of a sixth embodiment of a scaffold 1f according to the present invention. In this example the inner portion 2f comprises just one wall 4f (but it will be understood that it could also include a second wall, as in other embodiments). The inner portion 2f is triangular, and therefore defines a triangular channel 6f. The inner portion 2f further comprises axial conduits 120f, in this example three axial conduits 120f, respectively positioned at each corner of the triangular channel 6f. The axial conduits 120f may be made from the same material as the wall 4f. The axial conduits 120f allow for transport of materials, where these materials could be different materials to the materials travelling through the triangular channel 6f or the cavity 20f.

The outer portion 14f comprises a first wall 16f and a second wall 17f which are spaced apart, i.e. have a gap between them. The second set of walls 16f, 17f substantially surrounds the first wall 4f, as seen in the view from above of FIG. 12. Struts 24f extend between the first wall 16f and the second wall 17f of the outer portion 14f. The scaffold 1f further comprises a porous mesh base portion 28f, arranged to support a filler material placed inside the cavity 20f of the scaffold 1f.

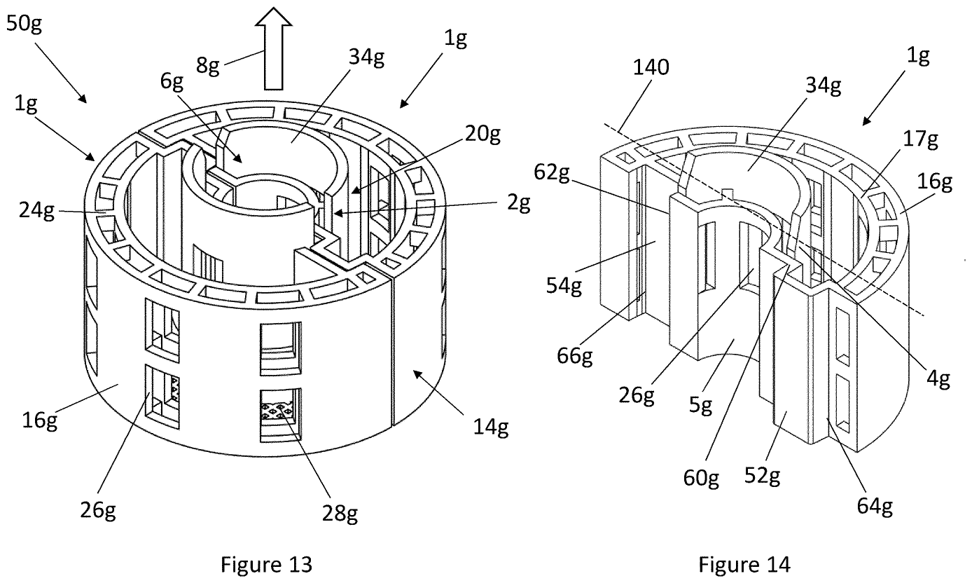

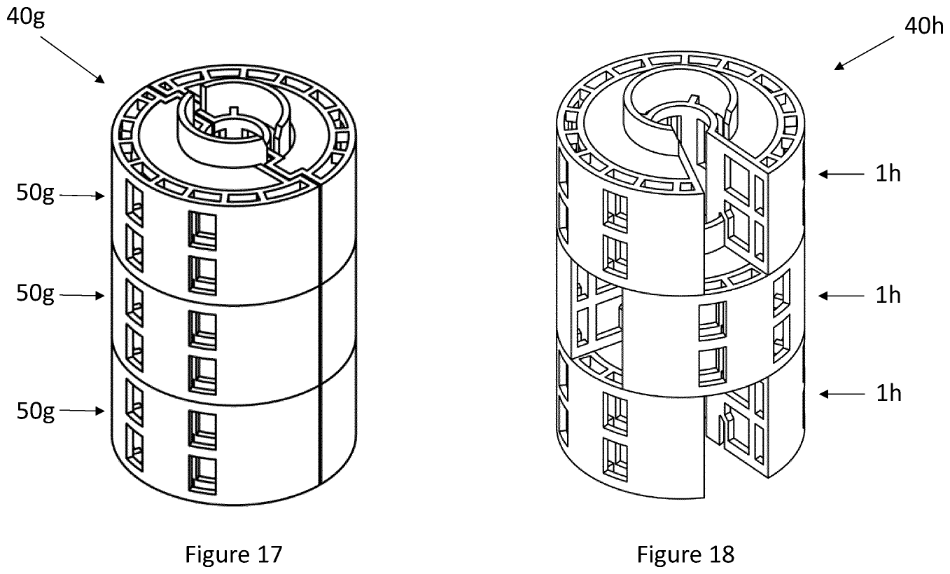

FIGS. 13-16 show various views of a seventh embodiment of a scaffold 1g according to a first aspect of the present invention, and also in accordance with a first embodiment of third and fourth aspects of the invention. FIGS. 13, 15 and 16 show a scaffold structure 50g assembled from two such scaffolds 1g. FIG. 17 shows a tissue engineering scaffold assembly 40g, assembled by axially interlocking three scaffold structures 50g, each formed from two radially interlocked scaffolds 1g. The same reference numerals as FIGS. 1-6 are used for corresponding components of the scaffold, followed by a “g”, rather than an “a”.

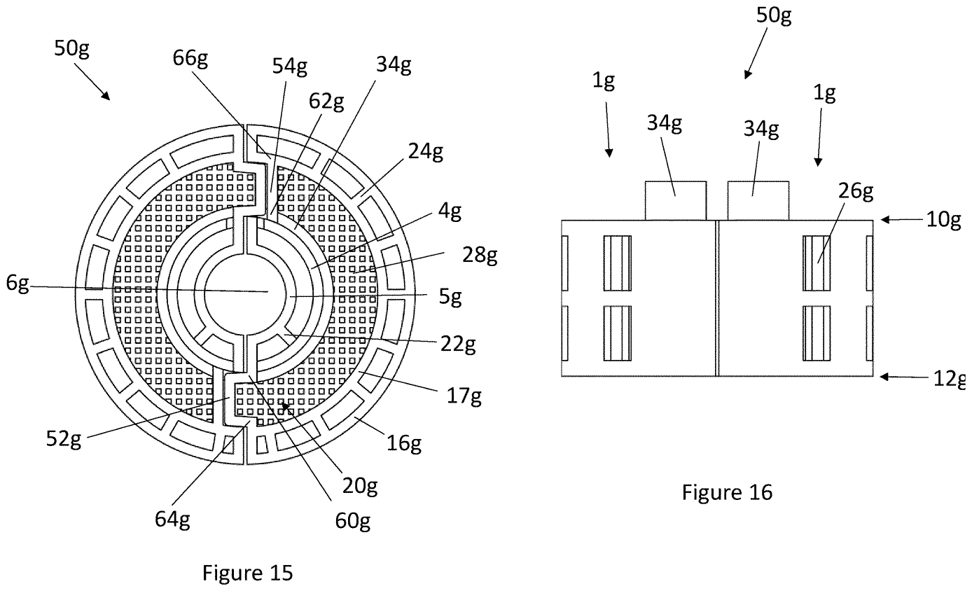

Each of the two tissue engineering scaffolds 1g comprises an inner portion 2g comprising a first wall 4g and a second wall 5g (seen in FIG. 15) which are spaced apart, i.e. have a gap between them. The inner portion 2g defines a channel 6g, which extends along an axial direction 8g from a first end 10g of the scaffold 1g to a second end 12g of the scaffold 1g.

The scaffold 1g further includes an outer portion 14g, which comprises a first wall 16g and a second wall 17g which are spaced apart, i.e. have a gap between them. The second set of walls 16g, 17g substantially surrounds the first set of walls 4g, 5g, as seen in the view from above of FIG. 15. In this embodiment both sets of walls are circular in shape, and extend around an angular range of 180 degrees, giving the scaffold a semi-circular cross section, along the axial direction, as seen in FIG. 15. Thus the channel 6g defined by the inner portion 2g has a semi-circular cross-section and is open in the radial direction, i.e. perpendicular to the axial direction. The spacing between the first and second sets of walls defines a cavity 20g, which again is semi-circular.

For clarity in FIG. 15 only the components of one of the two scaffolds 1g have been labelled with reference numerals, but it will be appreciated that the two scaffolds 1g are identical and therefore each of the components is present in both of the scaffolds 1g.

There are struts 22g, 24g, extending respectively between the first wall 4g, 16g, and the second wall 5g, 17g, of each of the inner and outer portions 2g, 14g. As seen in the Figures, the struts 22g, 24g are spaced apart in both the axial direction and in the circumferential direction, thereby allowing flow of material in both the axial and circumferential directions.

All four walls of the walls 4g, 5g, 16g, 17g are made of a solid, non-porous material. Each of the walls contains apertures 26g, which are open in the radial direction and which allow radial flow into and out of the cavity 20g and the channel 6g.

The scaffold 1g further comprises a base portion 28g, which is positioned at the second end 12g of the scaffold 1g. The base portion 28g is a porous mesh material which extends between the inner portion 2g and the outer portion 14g. There is a window in the base portion 28g such that it does not extend into the channel 6g, as seen in FIG. 15. The base portion 28g is arranged so that it can support a filler material (not shown) which can be placed inside the cavity 20g. The filler material can be seeded with cells and other materials suitable for growing bone in the cavity.

The scaffold 1g further includes a first axial interlocking part 34g which is arranged at the first end 10g of the scaffold 1g, and extends in the axial direction 8g beyond the walls 16g, 17g of the outer portion 14g. The first axial interlocking part 34g extends as part of the inner portion 2g, in particular as an extension of the first wall 4g, as seen in the perspective view of FIG. 14.

If a cross-section of the scaffold 1g were to be taken along the line 140 shown in FIG. 14, its overall structure would look substantially the same as the cross-section shown in FIG. 5. Thus, the scaffold 1g also includes a second axial interlocking part (not visible in FIG. 14) arranged at the second end 12g of the scaffold 1g. In particular, the second axial interlocking part is a semi-circular groove or recess, formed between the first wall 4g and the second wall 5g of the inner portion 2g. The first axial interlocking part 34g is a half-cylinder, which is sized to be received into the groove of the second axial interlocking part of another corresponding scaffold.

The scaffold 1g further comprises a first radial interlocking part 52g, which in the illustrated example is a tooth, extending axially along the length of the scaffold, and a second radial interlocking part 54g, which in the illustrated example is a groove, extending axially along the length of the scaffold. The second radial interlocking part 54g is arranged to engage with the first radial interlocking part 52g of another corresponding tissue engineering scaffold 1g to create an interlock in the radial direction, perpendicular to the axial direction.

In the illustrated example, the first and second radial interlocking parts 52g, 54g are formed to be connecting portions which each extend between an edge 60g, 62g of an opening in the inner portion and an edge 64g, 66g, of an opening in the outer portion.

As shown in FIGS. 13 and 15-17, these radial interlocking parts 52g, 54g allow two scaffolds 1g to be interlocked radially together, forming a scaffold structure 50g which spans an angular range of 360 degrees and forms a closed shape (in this case a closed circle). Multiple of these scaffold structures 50g can then be interlocked axially using their respective first and second axially interlocking parts to form a tissue engineering scaffold assembly 40g, as shown in FIG. 17. The arrangement of the first and second axially interlocking parts allows the scaffolds structures 40g to be interlocked so as to sit flush against one another in the axial direction, as seen in FIG. 17, giving a continuous structure, i.e. continuous inner and outer portions, channel and cavity.

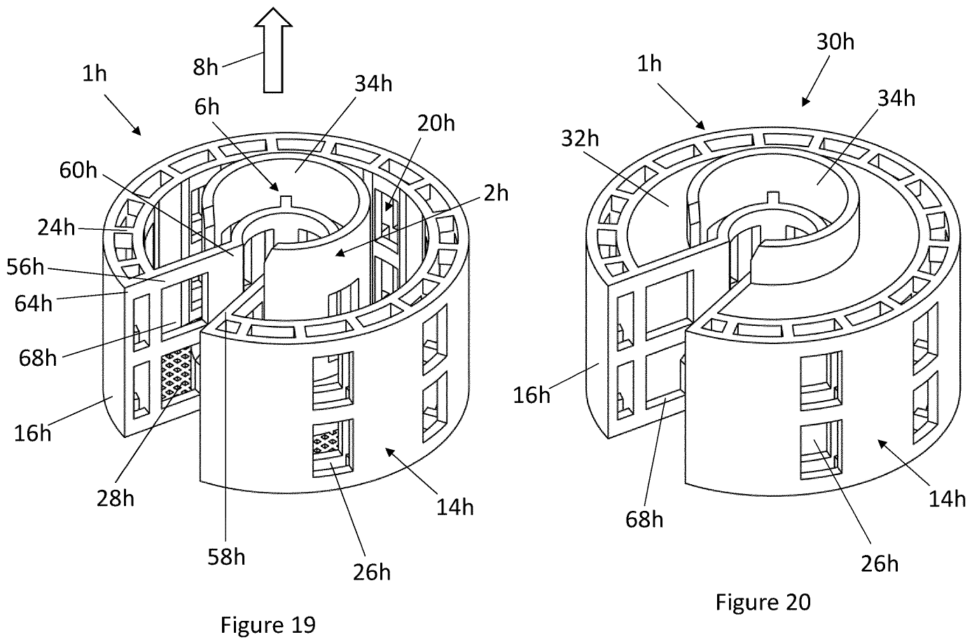

FIG. 18 shows another tissue engineering scaffold assembly 40h, according to a fourth embodiment of a second aspect of the present invention, which is made up of a plurality of scaffolds 1h according to an eighth embodiment of a first aspect of the present invention, and according to a second embodiment of a second aspect of the present invention. An individual scaffold 1h according to this example is shown in greater detail in FIGS. 19-22.

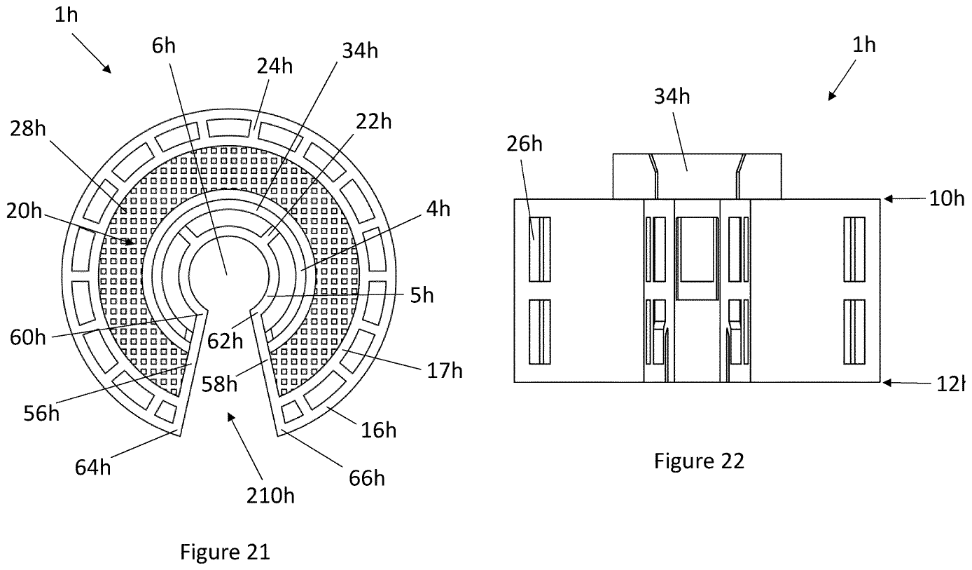

In this example, the tissue engineering scaffold 1h includes many of the same features as the scaffolds already described, which are labelled with like reference numerals, but now followed by an “h”. In particular, the scaffold 1h comprises an inner portion 2h comprising a first wall 4h and a second wall 5h (seen in FIG. 21), which are spaced apart, i.e. have a gap between them. The inner portion 2h defines a channel 6h, which extends along an axial direction 8h from a first end 10h of the scaffold 1h to a second end 12h of the scaffold 1h. The walls 4h, 5h of the inner portion extend around an axial range of less than 360 degrees, as seen in FIG. 21, such that the central channel 6h is open.

The scaffold 1h further includes an outer portion 14h, which comprises a first wall 16h and a second wall 17h. The second set of walls 16h, 17h substantially surrounds the first set of walls 4h, 5h, as seen in the view from above of FIG. 21. In this embodiment both sets of walls are circular in shape, and extend around an angular range of around 330 degrees, giving the scaffold a cross section which spans most of a circular cross-section, but contains an opening 210h. Since there are openings in both sets of walls, and these openings are aligned, the channel 6h defined by the inner portion 2h is open in the radial direction, i.e. perpendicular to the axial direction, as seen in FIGS. 19 and 20. The spacing between the first and second sets of walls defines a cavity 20h.

There are struts 22h, 24h, extending respectively between the first wall 4h, 16h, and the second wall 5h, 17h, of each of the inner and outer portions 2h, 14h. As seen in the Figures, the struts 22h, 24h are spaced apart in both the axial direction and in the circumferential direction, thereby allowing flow of material in both the axial and circumferential directions.

All four walls of the walls 4h, 5h, 16h, 17h are made of a solid, non-porous material. Each of the walls contains apertures 26h, which are open in the radial direction and which allow radial flow into and out of the cavity 20h and the channel 6h.

The scaffold 1g further comprises a base portion 28h, which is positioned at the second end 12h of the scaffold 1h. The base portion 28h is a porous mesh material which extends between the inner portion 2h and the outer portion 14h. There is a window in the base portion 28h such that it does not extend into the channel 6h, as seen in FIG. 21.

The base portion 28h is arranged so that it can support a filler material 32h which can be placed inside the cavity 20h. The filler material 32h can be seeded with cells and other materials suitable for growing bone in the cavity. A tissue engineering device 30h comprised of the scaffold 1h, and a filler material 32h placed within the cavity can be seen in FIG. 20.

The scaffold 1h further includes a first axial interlocking part 34h which is arranged at the first end 10h of the scaffold 1h, and extends in the axial direction 8h beyond the walls 16h, 17h of the outer portion 14h. The first axial interlocking part 34h extends as part of the inner portion 2h, in particular as an extension of the first wall 4h, as seen in FIG. 19. The scaffold also includes a second axial interlocking part (not shown) the same as that seen in the scaffold of FIG. 5.

The scaffold 1h further comprises a first connecting portion 56h and a second connecting portion 58h, extending across the channel 20h. The first connecting portion 56h extends between a first edge 60h of an opening in the inner portion 2h and a first edge 64h of an opening in the outer portion 14h. The second connecting portion 58h extends between a second edge 62h of an opening in the inner portion 2h and a second edge 66h of an opening in the outer portion 14h. The connecting portions 56h, 58h also include apertures 68h, as seen in FIGS. 19 and 20.

Although FIGS. 18-22 illustrate the scaffolds 1h of the tissue engineering scaffold assembly 40 has a single part having an angular range of about 300 degrees, it will be appreciated that two (or more) of the radially interlocked scaffolds 1g as previously described in relation to FIGS. 13-16 may instead be provided with a suitable angular range to achieve the opening shown in FIGS. 18-22 when radially interlocked together, i.e. each scaffold 1h may instead be provided by a scaffold structure comprising two or more scaffolds, radially interlocked together.

It will be appreciated by those skilled in the art that the invention has been illustrated by describing one or more specific embodiments thereof, but is not limited to these embodiments; many variations and modifications are possible, within the scope of the accompanying claims.

1. A tissue engineering scaffold comprising:
an inner portion comprising a first set of one or more walls defining a channel extending along an axial direction from a first end of the scaffold to a second end of the scaffold;
an outer portion comprising a second set of one or more walls and arranged such that the second set of one or more walls substantially surrounds the first set of one or more walls with a spacing between the first and second sets of one or more walls defining a cavity between the inner portion and the outer portion;
a first axial interlocking part arranged at the first end of the scaffold, extending in the axial direction beyond the first set of one or more walls and/or the second set of one or more walls; and
a second axial interlocking part arranged at the second end of the scaffold to engage with the first axial interlocking part of another corresponding tissue engineering scaffold to create an interlock in the axial direction.

2. A tissue engineering scaffold assembly comprising:
a plurality of corresponding tissue engineering scaffolds each according to the tissue engineering scaffold of claim 1;
wherein the second axial interlocking part of one of the plurality of corresponding tissue engineering scaffolds is engaged with the first axial interlocking part of another of the plurality of corresponding tissue engineering scaffolds to create an interlock in the axial direction.

3. The tissue engineering scaffold of claim 1, wherein the first and second sets of one or more walls extend across an angular range of less than 360 degrees, such that there is an opening in the first set of walls and in the second set of walls.

4. The tissue engineering scaffold of claim 3, wherein the opening in the first set of walls and the opening in the second set of walls are aligned.

5. The tissue engineering scaffold of claim 3, wherein the first and second sets of one or more walls extend across an angular range of more than 270 degrees.

6. (canceled)

7. (canceled)

8. The tissue engineering scaffold of claim 1, wherein the first set of one or more walls, or the second set of one or more walls comprise the first axial interlocking part and the second axial interlocking part.

9. The tissue engineering scaffold of claim 1, wherein the second axial interlocking part is sized to entirely accommodate the portion of the first axial interlocking part which extends in the axial direction beyond the first set of one or more walls and/or the second set of one or more walls, such that when interlocked in the axial direction the tissue engineering scaffold will be flush with another corresponding tissue engineering scaffold to which it is interlocked.

10. The tissue engineering scaffold of claim 1, wherein the tissue engineering scaffold further comprises a first radial interlocking part and a second radial interlocking part arranged to engage with the first radial interlocking part of another corresponding tissue engineering scaffold to create an interlock in the radial direction, perpendicular to the axial direction.

11. (canceled)

12. The tissue engineering scaffold of claim 3, wherein the tissue engineering scaffold further comprises a connecting portion, extending along the axial direction, and connecting the outer portion and the inner portion, such that an edge of the opening of the inner portion is connected to an edge of the opening of the outer portion by the connecting portion.

13. The tissue engineering scaffold of claim 1, wherein the first set of one or more walls and/or the second set of one or more walls are solid, non-porous walls, comprising one or more apertures, wherein the apertures comprise a chamfered or bevelled edge.

14. (canceled)

15. The tissue engineering scaffold of claim 1 wherein the inner portion comprises a first wall and a second wall, wherein the first wall and the second wall are concentric; and/or
wherein the outer portion comprises a first wall and a second wall, wherein the first wall and the second wall are concentric.

16. The tissue engineering scaffold of claim 15, wherein the tissue engineering scaffold further comprises struts, extending between the first wall and the second wall, wherein the struts are spaced apart in both the axial direction and in the circumferential direction, thereby allowing flow in both the axial and circumferential directions.

17. (canceled)

18. (canceled)

19. The tissue engineering scaffold of claim 1, wherein the outer portion is circular and the inner portion is polygonal.

20. The tissue engineering scaffold of claim 19, wherein the inner portion comprises axial conduits, arranged at each corner of the polygonal shape.

21. The tissue engineering scaffold of claim 1, wherein the length of the scaffold along the axial direction, between the first end and the second end, is approximately equal to, or less than, the width or diameter of the scaffold, perpendicular to the axial direction.

22. A tissue engineering device, comprising a tissue engineering scaffold as claimed in claim 1, and further comprising a filler material, positioned within the cavity of the tissue engineering scaffold.

23. A tissue engineering scaffold comprising:
an inner portion comprising a first set of one or more walls defining a channel extending along an axial direction from a first end of the scaffold to a second end of the scaffold;
an outer portion comprising a second set of one or more walls and arranged such that the second set of one or more walls substantially surrounds the first set of one or more walls with a spacing between the first and second sets of one or more walls defining a cavity between the inner portion and the outer portion;
wherein the first and second sets of one or more walls extend across an angular range of more than 270 degrees and less than 360 degrees, and wherein the opening in the first set of walls and the opening in the second set of walls are aligned, such that the channel is open in a direction other than the axial direction.

24. The tissue engineering scaffold of claim 23, further comprising a first axial interlocking part arranged at the first end of the scaffold, extending in the axial direction beyond the first set of one or more walls and/or the second set of one or more walls; and
a second axial interlocking part arranged at the second end of the scaffold to engage with the first axial interlocking part of another corresponding tissue engineering scaffold to create an interlock in the axial direction.

25. A tissue engineering scaffold comprising:
an inner portion comprising a first set of one or more walls defining a channel extending along an axial direction from a first end of the scaffold to a second end of the scaffold;
an outer portion comprising a second set of one or more walls and arranged such that the second set of one or more walls substantially surrounds the first set of one or more walls with a spacing between the first and second sets of one or more walls defining a cavity between the inner portion and the outer portion;
wherein the inner portion and/or the outer portion comprise more than one concentric wall.

26. A tissue engineering scaffold comprising:
an inner portion comprising a first set of one or more walls defining a channel extending along an axial direction from a first end of the scaffold to a second end of the scaffold;
an outer portion comprising a second set of one or more walls and arranged such that the second set of one or more walls substantially surrounds the first set of one or more walls with a spacing between the first and second sets of one or more walls defining a cavity between the inner portion and the outer portion;
wherein the second set of one or more walls extends across an angular range of less than 360 degrees; and
wherein the tissue engineering scaffold comprises a first radial interlocking part arranged to engage with the second radial interlocking part of another corresponding tissue engineering scaffold to create an interlock in the radial direction, perpendicular to the axial direction.
